(19) United States
(12) Patent Application Publication
(10) Pub. No.: US 2026/0162807 A1
(43) Pub. Date: Jun. 11, 2026

(54) SYSTEMS AND METHODS FOR LEARNING AN OPEN FOUNDATION MODEL IN MEDICAL IMAGING

(71) Applicant: Arizona Board of Regents on behalf of Arizona State University, Scottsdale, AZ (US)

(72) Inventors: Jianming Liang, Scottsdale, AZ (US); Mohammad Reza HOSSEINZADEH TAHER, Tempe, AZ (US); DongAo MA, Tempe, AZ (US)

(73) Assignee: Arizona Board of Regents on behalf of Arizona State University, Scottsdale, AZ (US)

(21) Appl. No.: 19/264,583

(22) Filed: Jul. 9, 2025

Related U.S. Application Data
(60) Provisional application No. 63/668,997, filed on Jul. 9, 2024.

(51) Int. Cl. G16H 30/40; G06N 3/0895; G06N 3/09; G06V 10/774

(52) U.S. Cl. CPC G16H 30/40; G06N 3/0895; G06N 3/09; G06V 10/774

(57) ABSTRACT

Learning a foundation model for medical images involves receiving a plurality of unlabeled medical images from a first plurality of patients, learning anatomical structures of the first plurality of patients via a self-supervised learning (SSL) framework from the plurality of unlabeled medical images, receiving a plurality of heterogeneously labeled medical images from a second plurality of patients, learning disease patterns of the second plurality of patients via a supervised learning method from the plurality of heterogeneously labeled medical images, and training the foundation model in medical imaging using the learned anatomical structures and the learned disease patterns via cyclical training.

### CLAIM OF PRIORITY

This application claims the benefit of U.S. Provisional Patent Application No. 63/668,997, filed Jul. 9, 2024, entitled “TOWARDS OPEN FOUNDATION MODELS IN MEDICAL IMAGING”, the disclosure of which is incorporated by reference herein in its entirety. This application is related to U.S. Non-Provisional application Ser. No. 18/627,810 (Attorney Docket No. 37684.683), entitled “SYSTEMS, METHODS, AND APPARATUSES FOR FOUNDATION MODELS LEARNED FROM ANATOMY IN MEDICAL IMAGING VIA SELF-SUPERVISION”; U.S. Non-Provisional application Ser. No. 19/064,520 (Attorney Docket No. 37684.699), entitled “SYSTEMS, METHODS, AND APPARATUSES FOR HIERARCHICAL EMBEDDINGS WITH LOCALIZABILITY, COMPOSABILITY AND DECOMPOSABILITY LEARNED FROM ANATOMY”; U.S. Non-Provisional application Ser. No. 18/825,923, entitled “SYSTEMS AND METHODS FOR LEARNING ANATOMICALLY CONSISTENT EMBEDDING FOR CHEST RADIOGRAPHY”; U.S. Non-Provisional application Ser. No. 19/057,881 (Attorney Docket No. 37684.6100), entitled “SYSTEMS, METHODS, AND APPARATUSES FOR ANATOMICALLY CONSISTENT EMBEDDINGS IN COMPOSITION AND DECOMPOSITION”; U.S. Non-Provisional application Ser. No. 19/059,165 (Attorney Docket No. 37684.6101), entitled “SYSTEMS, METHODS, AND APPARATUSES FOR LEARNING ANATOMICAL CONSISTENCY, SUB-VOLUME SPATIAL RELATIONSHIPS AND FINE-GRAINED APPEARANCE FOR COMPUTED TOMOGRAPHY”; U.S. Non-Provisional application Ser. No. 19/207,215 (Attorney Docket No. 37684.6102), entitled “SYSTEMS, METHODS, AND APPARATUSES FOR LEARNING ANATOMICAL CONSISTENCY, SUB-VOLUME SPATIAL RELATIONSHIPS AND FINE-GRAINED APPEARANCE FOR COMPUTED TOMOGRAPHY IMAGES”; U.S. Non-Provisional application Ser. No. 18/627,831 (Attorney Docket No. 37684.684), entitled “SYSTEMS, METHODS, AND APPARATUSES FOR ACCRUING AND REUSING KNOWLEDGE (ARK) FOR SUPERIOR AND ROBUST PERFORMANCE BY A TRAINED AI MODEL FOR USE WITH MEDICAL IMAGE CLASSIFICATION”; U.S. Non-Provisional application Ser. No. 19/205,846 (Attorney Docket No. 37684.6103), entitled “SYSTEMS, METHODS, AND APPARATUSES FOR DISENTANGLING ANATOMICAL VISUAL INFORMATION FROM DISEASES FOR LEARNING ENTANGLED REPRESENTATION”; U.S. Provisional Application No. 63/742,358 (Attorney Docket No. 37684.6107P), entitled “AUTODIDACTIC DENSE ANATOMICAL MODELS IN WHICH A SELF SUPERVISED LEARNING FRAMEWORK LEARNS TO ENCODE INHERENT PART WHOLE HIERARCHIES WITHIN MEDICAL IMAGES”; U.S. Provisional Application No. 63/670,543 (Attorney Docket No. 37684.6105P), entitled “ANATOMICALLY CONSISTENT EMBEDDINGS VIA COMPOSITION AND DECOMPOSITION”; U.S. Provisional Application No. 63/812,725 (Attorney Docket No. 37684.6117P), entitled “LAMPS: LEARNING ANATOMY FROM MULTIPLE PERSPECTIVES VIA SELF-SUPERVISION IN CHEST RADIOGRAPHS”; U.S. Provisional Application No. 63/781,892 (Attorney Docket No. 37684.6110P), entitled “METHOD AND APPARATUS FOR ACCRUING AND REUSING KNOWLEDGE FOR SUPERIOR AND ROBUST FOUNDATION MODELS”; U.S. Provisional Application No. 63/744,744 (Attorney Docket No. 37684.6108P), entitled “INTEGRATING CLASSIFICATION, LOCALIZATION, AND SEGMENTATION THROUGH LOCK-RELEASE PRETRAINING STRATEGY FOR CHEST X-RAY ANALYSIS”; and U.S. Provisional Application No. 63/690,726 (Attorney Docket No. 37684.6106P), entitled “AN OPEN FOUNDATION MODEL FOR CHEST RADIOGRAPHY”, the disclosures of which are incorporated by reference herein in their entireties.

### GOVERNMENT RIGHTS AND GOVERNMENT AGENCY SUPPORT NOTICE

This disclosure was made with government support under R01 HL128785 awarded by the National Institutes of Health. The government has certain rights in the disclosure.

### COPYRIGHT NOTICE

This document contains material which is subject to copyright protection. The copyright owner has no objection to the facsimile reproduction by anyone of the document as it appears in the Patent and Trademark Office records, but otherwise reserves all copyright rights whatsoever.

### TECHNICAL FIELD

The disclosed embodiments relate to learning an open foundation model for medical images, using a self-supervised learning (SSL) framework to learn anatomical structures in unlabeled medical images and a supervised learning framework to learn disease patterns in labeled medical images.

### BACKGROUND

Artificial intelligence (AI) models trained on broad data at scale that are adaptable to a diverse range of downstream tasks may be referred to as foundation models. These models can serve as a robust feature extractor. For example, in natural language processing (NLP), these models can extract embeddings including meaningful, semantics-rich, numerical vectors. Transforming words to their embeddings represents a major breakthrough for NLP and is the foundation of modern large language models (LLMs) such as ChatGPT-4, Gemini and Llama-3. These LLMs are generally trained via self-supervised learning (SSL) methods to capture the meaning of words and the linguistic structure of natural languages.

Several SSL methods have been developed for computer vision, and particularly medical imaging. However, their performance in the field of medical imaging does not match the performance of the above-mentioned LLMs in NLP because they lack the capability to capture the foundation of medical imaging, such as understanding the anatomical structures or patterns of the human body and recognizing disease patterns in humans and extracting such into embedding vectors for use in training AI models.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 illustrates how human vision easily organizes images into a tree-like structure, understanding the hierarchical relationships between objects and their parts.

FIG. 2 illustrates aspects of embodiments of the invention.

FIG. 3 depicts aspects of embodiments of the invention that learn, via an SSL framework, hierarchical anatomical structures of humans and provide discriminative features for different landmarks.

FIG. 4 illustrates the disclosed embodiments preserve composability and decomposability of anatomical structures where the embedding vector of a whole structure is equal to or close to the aggregate of the embeddings of its parts.

FIG. 5 further illustrates the disclosed embodiments preserve composability and decomposability of anatomical structures where the embedding vector of a whole structure is equal to or close to the aggregate of the embeddings of its parts.

FIG. 6 depicts disclosed embodiments for fully supervised learning using a student-teacher model with multi-task heads and trained via cyclic pretraining, aiming to accrue and reuse expert knowledge embedded in the heterogeneous labels with numerous public datasets.

### DETAILED DESCRIPTION

The disclosed embodiments provide a computer-implemented method and system for learning a foundation model for medical images. The disclosed embodiments obtain a plurality of unlabeled medical images from a first group of patients and learn anatomical structures of this first group of patients via a self-supervised learning (SSL) framework from the plurality of unlabeled medical images. The disclosed embodiments further obtain a plurality of heterogeneously labeled medical images from a second group of patients and learn disease patterns of this second group of patients via a supervised learning method from the plurality of heterogeneously labeled medical images. The disclosed embodiments then train the foundation model in medical imaging using the learned anatomical structures and the learned disease patterns via a cyclical training process. Further description of the disclosed embodiments follows.

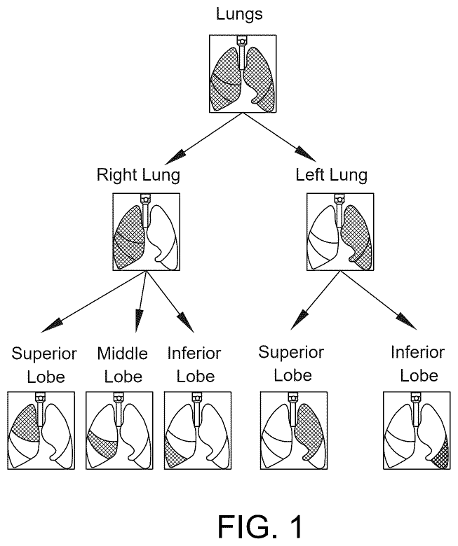

FIG. 1 illustrates how human perception effortlessly organizes objects into hierarchies to understand their part-whole relationships in images. Taking lungs as an example in FIG. 1, a lay person can form a hierarchy of the right and left lungs, and a radiologist can further see the lobes in sub hierarchies. To emulate this ability, the disclosed embodiments introduce a self-supervised learning (SSL) framework that explicitly learns to encode inherent part whole hierarchies within medical images into an embedding space, leading to the development of a powerful model that is foundational to medical imaging that can transform each pixel in medical images, for example, chest radiographs, into semantically meaningful embeddings, in which different anatomical structures are associated with distinct embeddings, and the same anatomical structures have nearly identical embeddings across patients. The disclosed embodiments receive a plurality of unlabeled medical images from a first group of patients and learn anatomical structures via the SSL framework from the plurality of unlabeled medical images, for example, by learning whole-part hierarchies of anatomical patterns in the plurality of unlabeled medical images.

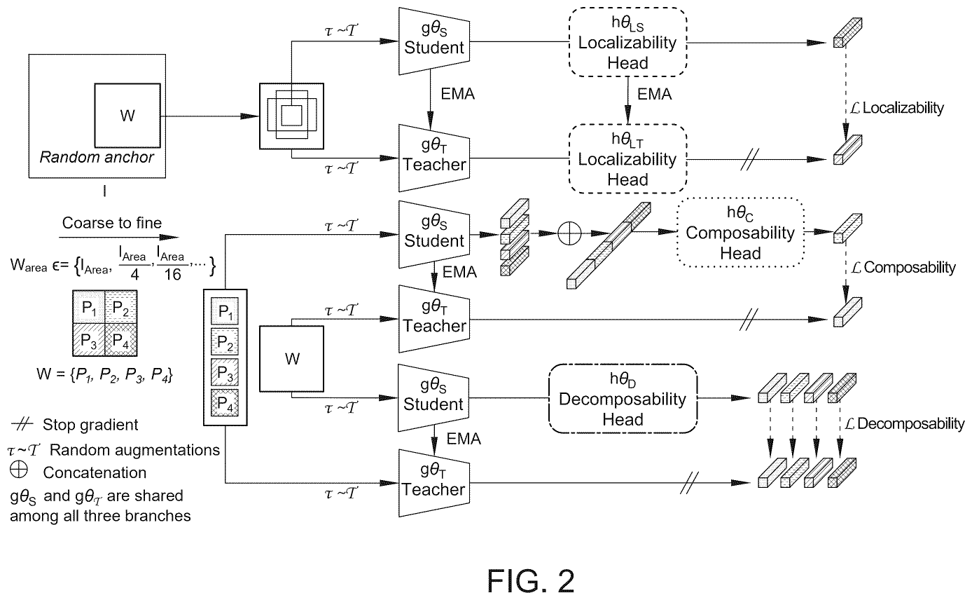

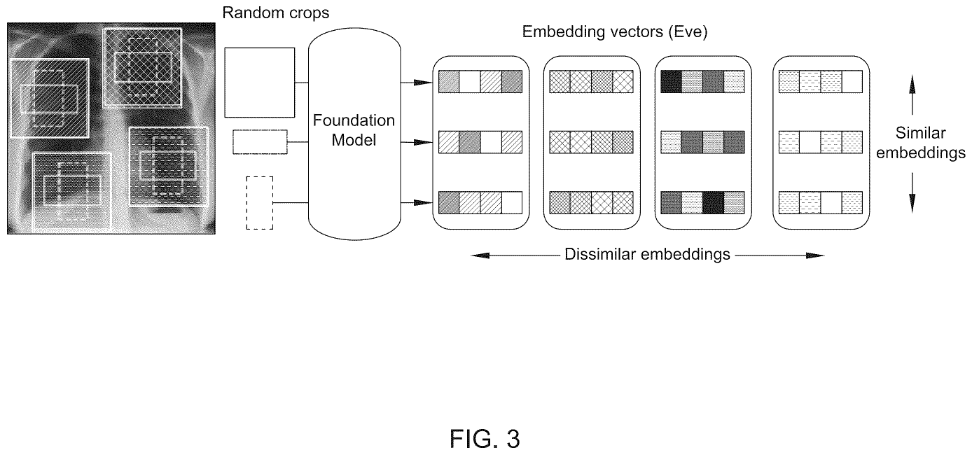

With reference to FIGS. 2 and 3, the disclosed embodiments learn anatomical structures via the SSL framework from the plurality of unlabeled medical images, for example, by learning where each anatomical structure is morphologically distinct from other anatomical structures in the anatomical patterns via a localizability branch of the SSL framework. For example, learning where each anatomical structure is morphologically distinct from other anatomical structures in the anatomical patterns via the localizability branch of the SSL framework may involve learning an embedding space where similar anatomical structures are clustered together and distinguished from dissimilar anatomical structures.

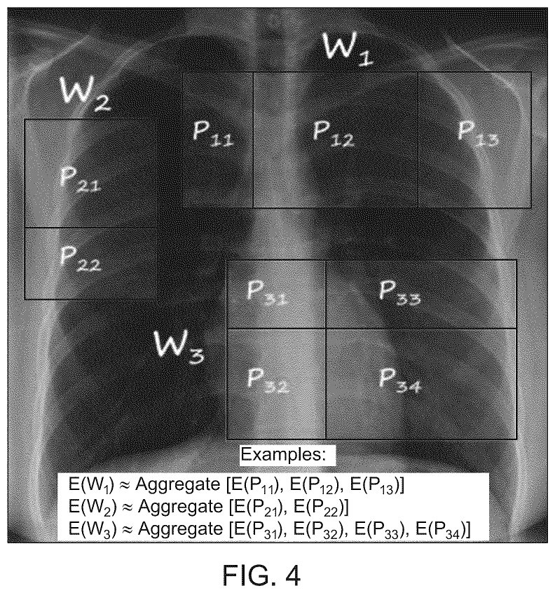

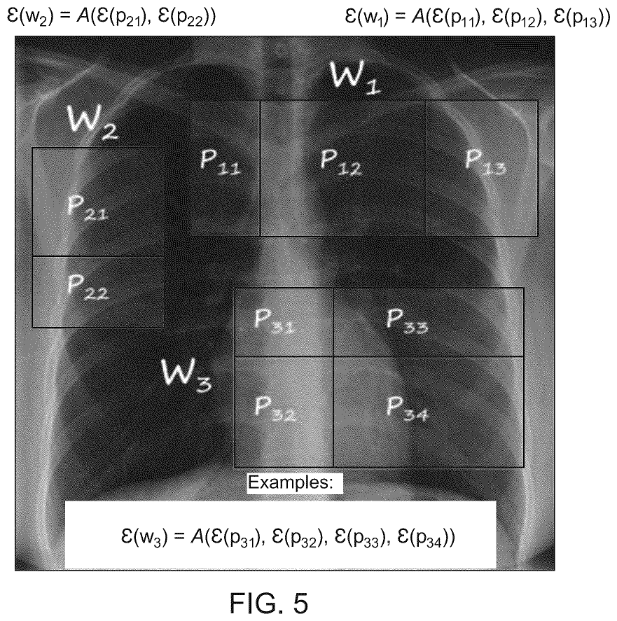

Additionally, with reference to FIGS. 2, 4, 5, according to the disclosed embodiments, learning anatomical structures via the SSL framework from the plurality of unlabeled medical images may involve learning where each smaller anatomical structure is an integrated part of a larger anatomical structure in the plurality of unlabeled medical images via a composability branch of the SSL framework. Further, learning anatomical structures via the SSL framework from the plurality of unlabeled medical images may involve, according to the disclosed embodiments, learning where each larger anatomical structure comprises a plurality of smaller anatomical structures via a decomposability branch of the SSL framework.

Thus, the framework presented in the disclosed embodiments, and illustrated in FIG. 2, learns hierarchical representations in a coarse-to-fine-manner via three branches: localizability, composability, and decomposability. Given an anchor whole w randomly sampled from an image I, the localizability branch augments and processes w and its multi-scale views, and enforces consistency between their embeddings, yielding distinct features for different anatomical structures. The composability branch decomposes w into a set of parts and enforces consistency between the embedding of w and the aggregated embeddings of its parts, encoding part-whole relations. The decomposability branch decomposes the embedding of w to acquire the embeddings of its constituent parts and enforce consistency between the embeddings of parts and their decomposed counterparts, capturing whole-part relations. As illustrated in FIG. 2, the disclosed embodiments can transform each pixel in medical images (e.g., chest radiographs) into semantically meaningful embeddings, where different anatomical structures (indicated by different groups or clusters of boxes) are associated with different embeddings, and the same anatomical structures have identical or nearly identical embeddings at all resolutions and scales (indicated by different box shapes) across patients.

As mentioned above, the framework comprises three branches: (1) localizability, which compels the model to learn a semantically structured embedding space by discriminating between different anatomical structures; (2) composability, which empowers the model to learn part-whole relations by constructing each anatomical structure through the integration of its constituent parts; and (3) decomposability, which encourages the model to learn whole-part relations by decomposing each anatomical structure into its constituent parts. Unifying these three branches together in a coarse-to-fine learning approach, the localizability branch enables the model to preserve harmony in embeddings of semantically similar anatomical structures in a hierarchy of scales. Simultaneously, composability and decomposability branches empower the model to not only convey hierarchical relationships but also preserve diversity of semantically similar anatomical structures across patients through encoding finer-grained anatomical information of their constituent parts. The disclosed embodiments represent a significant advancement from previous autodidactic dense anatomical models that learn autodidactically and yield dense anatomical embedding for semantic richness.

FIG. 3 illustrates the disclosed embodiments learning anatomical structures via the SSL framework from the plurality of unlabeled medical images by transforming patches of the plurality of unlabeled medical images into respective, semantics-rich anatomical embeddings. According to an embodiment, transforming patches of the plurality of unlabeled medical images into respective, semantics-rich anatomical embeddings may involve transforming patches comprising one or more pixels of the plurality of unlabeled medical images into respective, semantics-rich anatomical embeddings. According to an embodiment, transforming patches of the plurality of unlabeled medical images into respective, semantics-rich anatomical embeddings may involve transforming patches of the plurality of medical images relating to a selected anatomical structure into similar, respective, semantics-rich anatomical embeddings across the plurality of medical images. According to an embodiment, transforming patches of the plurality of medical images relating to the selected anatomical structure into similar, respective, semantics-rich anatomical embeddings across the plurality of medical images may involve transforming patches centered at a selected pixel in the plurality of medical images relating to the selected anatomical structure into similar, respective, semantics-rich anatomical embeddings across the plurality of medical images.

Currently, SSL methods have not proved capable of learning disease patterns in the human body given the vast number of diseases. Expert knowledge on diseases and disease patterns is essential. However, this expert knowledge is spread across multiple different datasets annotated by different experts using heterogenous labels or annotations. Thus, the disclosed embodiments take a different approach to learning disease patterns in the human body, instead of using an SSL method for learning anatomical structures or patterns in the human body.

For instance, the disclosed embodiments obtain a plurality of heterogeneously labeled medical images, for example, a plurality of different datasets each comprising a plurality of medical images for a second plurality of patients, and aggregate the heterogeneous annotations provided by a plurality of experts across the plurality of different datasets of medical images to form the plurality of heterogeneously labeled medical images. The disclosed embodiments then learn disease patterns of the second plurality of patients via a supervised learning method from the plurality of heterogeneously labeled medical images.

For example, a disclosed embodiment provides a framework that accrues, and reuses knowledge embedded in heterogeneous expert annotations across numerous datasets. The framework employs a student-teacher learning model with multi-task heads and trained via cyclic pretraining to accrue and reuse the expert knowledge embedded in the heterogeneous labels across the public datasets. One embodiment involves accruing and reusing information obtained from heterogeneously annotated labels associated with a plurality of medical image datasets to pretrain a model, by receiving the plurality of medical image datasets with the associated heterogeneously annotated labels, cyclically pretraining the model via a student encoder of the student-teacher learning model by iterating sequentially through the plurality of medical image datasets each round of pretraining to accrue knowledge from the associated heterogeneously annotated labels, and then updating a teacher encoder of the student-teacher learning model each round of pretraining based on the student encoder's accrued knowledge.

One disclosed embodiment involves receiving a plurality of image datasets (e.g., chest X-ray image datasets) each including one or more of a plurality of inconsistent or heterogeneous (expert-level) annotations across a corresponding one or more of a plurality of tasks including classification (identifying diseases), localization (generating bounding boxes), and segmentation (delineating boundaries) tasks employed in (medical) imaging. The disclosed embodiment trains the learning model using the plurality of image datasets to retain general knowledge across all the plurality of tasks while preventing overfitting to any one of the plurality of tasks, using an end-to-end framework comprising a student-teacher model, a shared backbone, a classification task branch, a localization task branch, and a segmentation task branch. According to the disclosed embodiment, the framework integrates and concurrently performs the plurality of tasks on the plurality of image datasets, including implementing a lock-release pretraining strategy that involves cyclically training the learning model using the plurality of image datasets and sequentially processing each of the plurality of image datasets.

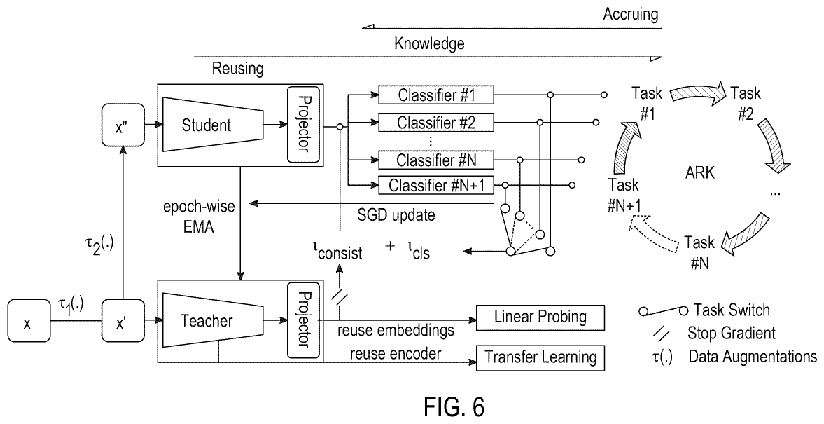

FIG. 6 is a functional diagram of a foundation model that accrues and reuses knowledge, termed Foundation ARK or simply ARK. ARK aims to aggregate numerous datasets with heterogeneous annotations to diversify patient population, accrue knowledge from diverse experts, and meet the demand by deep learning for massively annotated training data, offering superior and robust performance yet reducing annotation cost.

ARK is built on a student-teacher model with multi-task heads and trained via cyclic pretraining, aiming to accrue and reuse the expert knowledge embedded in the heterogeneous labels with numerous public datasets (see section 2 for details). Models pretrained with ARK may be referred to as Foundation ARK or, simply, ARK. ARK exhibits superior robustness over state of the art fully/self-supervised models in mitigating underdiagnosis and reducing gender-related biases, with lower false-negative rates and greater robustness to imbalanced data.

This performance enhancement is attributed to the observation that aggregating numerous public datasets costs nearly nothing but enlarges data size, diversifies patient populations, and accrues expert knowledge from many sources worldwide, thereby offering unprecedented performance yet reducing annotation cost. More importantly, ARK is fundamentally different from self-supervised learning (SSL) and federated learning (FL) in concept. SSL can naturally handle images from different sources, but their associated expert annotations are left out of pretraining. Every bit of expert annotation counts, conveying valuable knowledge. FL can utilize data with annotations from different sources, typically involving homogeneous labels, but it mainly concerns data privacy. By contrast, ARK focuses on heterogeneous expert annotations with public data with no concern for data privacy and employs centralized training, which usually offers better performance with the same amount of data and annotation than distributed training as in FL.

Embodiments of ARK provide for aggregating public datasets to enlarge and diversify training data, using a student-teacher model with multi-task heads via cyclic pretraining that accrues expert knowledge from existing heterogeneous annotations to achieve superior and robust performance yet reduce annotation cost.

ARK aims to learn superior and robust visual representations from large-scale aggregated medical images by accruing and reusing the expert knowledge embedded in all available heterogeneous labels. As for accruing knowledge into the student via cyclic pretraining, a significant challenge with training a single model using numerous datasets created for different tasks is label inconsistency (i.e., heterogeneity). Manually consolidating heterogeneous labels from different datasets would be a hassle. To circumvent this issue, for each task, a specific classifier, called task head, is introduced to learn from its annotation and encode the knowledge into the model. A task head can be easily plugged into ARK, making ARK scalable to additional tasks. With multi-task heads, ARK can learn from multiple tasks concurrently or cyclically. In concurrent pretraining, a mini-batch is formed by randomly sampling an equal number of images from each dataset, and the loss for each image is computed based on its associated dataset identifier (ID) and labels. This idea is intuitive, but the model hardly converges; it is suspected that the loss summation over all task heads simultaneously weakens gradients for back-propagation, causing confusion in weight updating. Embodiments opt for cyclic pre-training by iterating through all datasets sequentially in each round to accrue expert knowledge from all available annotations, a strategy that has been found to stabilize ARK's pretraining and accelerates its convergence.

One disclosed embodiment accrues knowledge into the teacher via epoch-wise exponential moving average (EMA). To further summarize the accrued knowledge and accumulate the learning experiences in the historical dimension, ARK is a teacher model that shares the same architecture with the student. The teacher is updated using (EMA) based on the student's one epoch of learning at the end of each task. Eventually, the expert knowledge embedded in all labels and all historical learning experiences are accrued in the teacher model for further reuse in the cyclic pretraining and for future application-specific target tasks.

ARK reuses accrued knowledge from the student to bolster cyclic pre-training. If the model learns from multiple tasks sequentially, it may “forget” the previously learned knowledge, and its performance on an old task may degrade catastrophically. This problem is addressed naturally in ARK by cyclic pretraining, where the model revisits all the tasks in each round and reuses all knowledge accrued from the previous rounds and tasks to strengthen its learning from the current and future tasks. That is, by regularly reviewing the accrued knowledge through task revisitation, ARK not only prevents forgetting but also enables more efficient and effective learning from multiple tasks iteratively.

ARK reuses accrued knowledge from the teacher to mitigate forgetting. To leverage the accumulated knowledge of the teacher model as an additional self-supervisory signal, a consistency loss between the student and the teacher is incorporated, as shown in FIG. 6. To enhance this supervision, projectors are introduced in ARK that map the outputs of the student and teacher encoders to the same feature space. This further reinforces the feedback loop between the student and teacher models, facilitating the transfer of historical knowledge from the teacher to the student as a reminder to mitigate forgetting.

Ark has a number of properties. Firstly, it is knowledge-centric. Annotating medical images by radiologists for deep learning is a process of transferring their in-depth knowledge and expertise in interpreting medical images and identifying abnormalities to a medium that is accessible for computers to learn. ARK's superior and robust performance is attributed to the accumulation of expert knowledge conveyed through medical imaging annotations from diverse expert sources worldwide. At the core of Ark is acquiring and sharing knowledge: “knowledge is power” (Mac Flecknoe) and “power comes not from knowledge kept but from knowledge shared” (Bill Gates). Second, ARK is label-agnostic, task-scalable and annotation-heterogeneous. ARK is label agnostic as it does not require prior label “understanding” of public datasets, but instead uses their originally provided labels. It is designed with pluggable multi-task heads and cyclic pretraining to offer flexibility and scalability for adding new tasks without manually consolidating heterogeneous labels or training task-specific controllers/adapters. Therefore, ARK intrinsically handles the annotation heterogeneity across different datasets. Third, ARK is application-versatile. ARK trains versatile foundation models by utilizing a large number of publicly available images from diverse sources and their readily accessible diagnostic labels. Indeed, ARK models are more robust, generalizable, and transferable to a wide range of application-specific target tasks across diseases (e.g., pneumothorax, tuberculosis, cardiomegaly) and anatomies (e.g., lung, heart, rib), highlighting ARK's versatility.

Finally, the disclosed embodiments train the foundation model in medical imaging using the learned anatomical structures and the learned disease patterns via cyclical training. Doing so trains the foundation model to be adaptable for a plurality of diverse downstream tasks in connection with analyzing medical images for new patients.

Embodiments o the disclosure contemplate a machine or system within which embodiments may operate, be installed, integrated, or configured. In accordance with one embodiment, the system includes at least a processor and a memory therein to execute instructions including implementing any application code to perform any one or more of the methodologies discussed herein. Such a system may communicatively interface with and cooperatively execute with the benefit of remote systems, such as a user device sending instructions and data, a user device to receive output from the system.

A bus interfaces various components of the system amongst each other, with any other peripheral(s) of the system, and with external components such as external network elements, other machines, client devices, cloud computing services, etc. Communications may further include communicating with external devices via a network interface over a LAN, WAN, or the public Internet.

In alternative embodiments, the system may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes code that implements the three branches of the SSL framework described herein, namely, the localizability branch, the composability branch, and the decomposability branch.

The processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. The processor may also be one or more special-purpose processing devices such as an application-specific integrated circuit (ASIC), a field programmable gate array (FPGA), a digital signal processor (DSP), network processor, or the like. The processor is configured to execute processing logic for performing the operations and functionality discussed herein.

The system may further include a network interface card. The system also may include a user interface (such as a video display unit, a liquid crystal display, etc.), an alphanumeric input device (e.g., a keyboard), a cursor control device (e.g., a mouse), and a signal generation device (e.g., an integrated speaker). According to an embodiment of the system, the user interface communicably interfaces with a user client device remote from the system and communicatively interfaces with the system via a public Internet.

The system may further include peripheral device (e.g., wireless or wired communication devices, memory devices, storage devices, audio processing devices, video processing devices, etc.).

A secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the system, the main memory and the processor also constituting machine-readable storage media. The software may further be transmitted or received over a network via the network interface card.

In addition to various hardware components depicted in the figures and described herein, embodiments further include various operations which are described herein. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the disclosure provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk, read-only memories (ROMs), random access memories (RAMs), EPROMs, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays which are described in greater detail herein. Various customizable and special purpose systems may be utilized in conjunction with specially configured programs in accordance with the teachings herein, or it may prove convenient, in certain instances, to construct a more specialized apparatus to perform the required method steps. The required structure for a variety of these systems will appear as set forth in the description. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A computer-implemented method for learning a foundation model for medical images, comprising:
receiving a plurality of unlabeled medical images from a first plurality of patients;
learning anatomical structures of the first plurality of patients via a self-supervised learning (SSL) framework from the plurality of unlabeled medical images;
receiving a plurality of heterogeneously labeled medical images from a second plurality of patients;
learning disease patterns of the second plurality of patients via a supervised learning method from the plurality of heterogeneously labeled medical images; and
training the foundation model in medical imaging using the learned anatomical structures and the learned disease patterns via cyclical training.

2. The computer-implemented method of claim 1 wherein learning anatomical structures via the SSL framework from the plurality of unlabeled medical images comprises learning anatomical structures via the SSL framework by learning whole-part hierarchies of anatomical patterns in the plurality of unlabeled medical images.

3. The computer-implemented method of claim 1, wherein learning anatomical structures via the SSL framework from the plurality of unlabeled medical images comprises learning where each anatomical structure is morphologically distinct from other anatomical structures in the anatomical patterns via a localizability branch of the SSL framework.

4. The computer-implemented method of claim 3, wherein learning where each anatomical structure is morphologically distinct from other anatomical structures in the anatomical patterns via the localizability branch of the SSL framework comprises learning an embedding space where similar anatomical structures are clustered together and distinguished from dissimilar anatomical structures.

5. The computer-implemented method of claim 1, wherein learning anatomical structures via the SSL framework from the plurality of unlabeled medical images comprises learning where each smaller anatomical structure is an integrated part of a larger anatomical structure in the plurality of unlabeled medical images via a composability branch of the SSL framework.

6. The computer-implemented method of claim 1, wherein learning anatomical structures via the SSL framework from the plurality of unlabeled medical images comprises learning where each larger anatomical structure comprises a plurality of smaller anatomical structures via a decomposability branch of the SSL framework.

7. The computer-implemented method of claim 1, wherein learning anatomical structures via the SSL framework from the plurality of unlabeled medical images comprises transforming patches of the plurality of unlabeled medical images into respective, semantics-rich anatomical embeddings.

8. The computer-implemented method of claim 7, wherein transforming patches of the plurality of unlabeled medical images into respective, semantics-rich anatomical embeddings comprises transforming patches comprising one or more pixels of the plurality of unlabeled medical images into respective, semantics-rich anatomical embeddings.

9. The computer-implemented method of claim 7, wherein transforming patches of the plurality of unlabeled medical images into respective, semantics-rich anatomical embeddings comprises transforming patches of the plurality of medical images relating to a selected anatomical structure into similar, respective, semantics-rich anatomical embeddings across the plurality of medical images.

10. The computer-implemented method of claim 9, wherein transforming patches of the plurality of medical images relating to the selected anatomical structure into similar, respective, semantics-rich anatomical embeddings across the plurality of medical images comprises transforming patches centered at a selected pixel in the plurality of medical images relating to the selected anatomical structure into similar, respective, semantics-rich anatomical embeddings across the plurality of medical images.

11. The computer-implemented method of claim 1, wherein receiving the plurality of heterogeneously labeled medical images comprises:
receiving a plurality of different datasets each comprising a plurality of medical images for the second plurality of patients; and
aggregating heterogeneous annotations provided by a plurality of experts across the plurality of different datasets of medical images to form the plurality of heterogeneously labeled medical images.

12. The computer-implemented of claim 1, wherein training the foundation model in medical imaging using the learned anatomical structures and the learned disease patterns via cyclical training comprises training the foundation model to be adaptable for a plurality of diverse downstream tasks in connection with analyzing medical images for a third plurality of patients.

13. A system comprising:
a memory to store instructions;
a processor to execute the instructions stored in the memory;
a receive interface to receive a plurality of unlabeled medical images from a first plurality of patients and to receive a plurality of heterogeneously labeled medical images from a
second plurality of patients;
wherein the system is configured to learn a foundation model for medical images by:
learning anatomical structures of the first plurality of patients via a self-supervised learning (SSL) framework from the plurality of unlabeled medical images;
learning disease patterns of the second plurality of patients via a supervised learning method from the plurality of heterogeneously labeled medical images; and
training the foundation model in medical imaging using the learned anatomical structures and the learned disease patterns via cyclical training.

14. The system of claim 13, wherein learning anatomical structures via the SSL framework from the plurality of unlabeled medical images comprises learning where each anatomical structure is morphologically distinct from other anatomical structures in the anatomical patterns via a localizability branch of the SSL framework.

15. The system of claim 13, wherein learning anatomical structures via the SSL framework from the plurality of unlabeled medical images comprises learning where each smaller anatomical structure is an integrated part of a larger anatomical structure in the plurality of unlabeled medical images via a composability branch of the SSL framework.

16. The system of claim 13, wherein learning anatomical structures via the SSL framework from the plurality of unlabeled medical images comprises learning where each larger anatomical structure comprises a plurality of smaller anatomical structures via a decomposability branch of the SSL framework.

17. The system of claim 13, wherein receiving the plurality of heterogeneously labeled medical images comprises:
receiving a plurality of different datasets each comprising a plurality of medical images for the second plurality of patients; and
aggregating heterogeneous annotations provided by a plurality of experts across the plurality of different datasets of medical images to form the plurality of heterogeneously labeled medical images.

18. A non-transitory computer-readable storage media having instructions stored thereupon that, when executed by a system having at least a processor and a memory therein, learn a foundation model for medical images, by executing the instructions via the processor for:
receiving a plurality of unlabeled medical images from a first plurality of patients;
learning anatomical structures of the first plurality of patients via a self-supervised learning (SSL) framework from the plurality of unlabeled medical images;
receiving a plurality of heterogeneously labeled medical images from a second plurality of patients;
learning disease patterns of the second plurality of patients via a supervised learning method from the plurality of heterogeneously labeled medical images; and
training the foundation model in medical imaging using the learned anatomical structures and the learned disease patterns via cyclical training.

19. The non-transitory computer-readable storage media of claim 18, wherein learning anatomical structures via the SSL framework from the plurality of unlabeled medical images comprises learning where each anatomical structure is morphologically distinct from other anatomical structures in the anatomical patterns via a localizability branch of the SSL framework.

20. The non-transitory computer-readable storage media of claim 18, wherein receiving the plurality of heterogeneously labeled medical images comprises:
receiving a plurality of different datasets each comprising a plurality of medical images for the second plurality of patients; and
aggregating heterogeneous annotations provided by a plurality of experts across the plurality of different datasets of medical images to form the plurality of heterogeneously labeled medical images.
